(19) United States
(12) Patent Application Publication
(10) Pub. No.: US 2025/0303171 A1
(43) Pub. Date: Oct. 2, 2025

(54) IMPLANTABLE STIMULATION SYSTEMS AND ENERGY-EFFICIENT SENSING METHODS

(71) Applicant: The Alfred E. Mann Foundation for Scientific Research, Valencia, CA (US)

(72) Inventors: Brian SHELTON, San Beaunaventura, CA (US); Hitesh HOTLANI, Santa Clarita, CA (US)

(21) Appl. No.: 18/935,250

(22) Filed: Nov. 1, 2024

Related U.S. Application Data
(60) Provisional application No. 63/570,505, filed on Mar. 27, 2024.

(51) Int. Cl. A61N 1/36; A61N 1/372; G16H 20/17; G16H 40/63

(52) U.S. Cl. CPC A61N 1/36139; A61N 1/36053; A61N 1/36057; A61N 1/36089; A61N 1/3611; A61N 1/36114; A61N 1/3615; A61N 1/36171; A61N 1/36175; A61N 1/37217; A61N 1/37247; G16H 20/17; G16H 40/63

(57) ABSTRACT

The present disclosure relates to stimulation systems, comprising, e.g., an implantable pulse generator or pump, biometric sensors incorporated therein, and one or more external sensors which may optionally be incorporated into one or more electronic devices (e.g., a wearable, portable, or stationary device). In some aspects, the one or more electronic devices may be configured to control one or more parameters of the stimulation systems.

### CROSS-REFERENCE TO RELATED APPLICATION

This application claims priority to U.S. Provisional Application No. 63/570,505, filed Mar. 27, 2024, the contents of which is hereby incorporated in its entirety.

### TECHNICAL FIELD

The present disclosure generally relates to stimulation systems and methods of using the same. In particular, the present disclosure relates to stimulation systems, comprising, e.g., an implantable pulse generator, biometric sensors incorporated therein, and one or more external sensors which may optionally be incorporated into one or more electronic devices.

### BACKGROUND

Various types of devices have been developed for implantation into the human body to provide various types of health-related therapies and/or monitoring. Examples of such devices, generally known as implantable medical devices (“IMDs”), include cardiac pacemakers, cardioverter/defibrillators, cardiomyostimulators, various physiological stimulators including nerve, muscle, and deep brain stimulators, various types of physiological monitors, and drug delivery systems, just to name a few. For the purposes of this application, reference will be made only to implantable neurostimulators, such as implantable pulse generators (“IPGs”), it being understood that the principles described herein may have applicability to other implantable medical devices as well.

IPGs are often used in the context of neuromodulation therapy, and, in general, comprise a hermetically sealed housing containing stimulation circuitry. Electrically conductive feedthrough pins extend from the stimulation circuitry and into a header connector, which is mounted upon the IPG housing, and the header connector defines a receptacle adapted to receive and couple with the proximal end of a stimulation lead.

Stimulation leads may be manufactured in a variety of configurations to accommodate different applications or anatomical targets. For example, a non-exhaustive list of stimulation leads known in the prior art includes: nerve cuffs as disclosed in U.S. Pat. No. 9,283,394; helical electrodes as disclosed in U.S. Pat. No. 4,573,481; paddle electrodes (i.e. electrode arrays) as disclosed in U.S. Pat. Pub. 20120209285A1; and linear electrodes (e.g. percutaneous electrodes) as disclosed in U.S. Pat. No. 8,650,747.

Many stimulation systems known in the prior art may include patient controllers, physician programming devices, and external chargers. Further, many implantable stimulation systems incorporate sensors for the purposes of modulating stimulation parameters, monitoring patient health or activity, and monitoring the effectiveness of treatment, among others. For example, biometric sensors such as, e.g., electrocardiograms, pulse oximeters, accelerometers, inertial measurement units (“IMUs”), gyroscopes, magnetometers, and/or audio transducers may be incorporated into implantable stimulation systems.

An implantable IMU, for example, can be used to detect multiple physiological parameters such as, e.g., respiration, heart rate, patient activity, patient mobility, or patient position, and this makes it a convenient addition to implantable medical devices. However, continuous sensing by IMD often requires a significant allocation of energy, which can contribute to battery drain or have an otherwise deleterious effect on the lifespan of an IMD. There are also other issues that can arise with implanted sensors. With respects to implanted IMUs, patient movement can overwhelm the implanted sensor, making it difficult for the device to detect biometric signals of relatively smaller amplitude, such as patient respiration or heart rate.

### SUMMARY OF SELECTED ASPECTS

The stimulation systems and methods described herein address various shortcomings in the art, e.g., by providing: an improved user interface and/or experience, enhanced functional sensing, and/or increased energy efficiency, compared to available stimulation systems and methods. In some aspects, one or more of these benefits are achieved by incorporating the use of an external electronic device (e.g., a wearable device, a bedside device, etc.) that may, e.g., function as a controller for the stimulation system and/or integrate one or more biometric or other sensors. In some aspects, use of an external electronic (e.g., wearable) device allows for the collection of additional sensor data that may be used to supplement (or replace) sensor data provided by any implanted, or other external, sensors available to the stimulation system. Moreover, the device may be used as an external control device, avoiding the need for a patient to have access to a custom-made dedicated controller, smart phone, or other means of control. Wearable devices, such as smart watches, provide a convenient form factor that is easily maintained by the subject (e.g., a wrist-worn watch is less likely to be lost or misplaced compared to a smart phone, dedicated controller, or other personal electronic device. Other form factors may also be used, e.g., any of the sensors or functional aspects of the wearable and other devices described herein may alternatively be integrated into devices that are primarily stationary, rather than worn by a user (e.g., a bedside device), as well as portable devices that may not be specifically intended as a wearable device (e.g., a handheld dedicated controller or smart phone). Such embodiments may lack some of the convenience of a wearable device, but offer other advantages, e.g., additional space for a larger battery and/or more sensors, additional processing capabilities, and/or the option to be powered from an outlet. Various other benefits will become apparent in view of the present description and the accompanying drawings.

For example, the stimulation systems described herein, and components or methods of use thereof, may be used to automatically optimize treatment parameters with limited need for input from a clinician. Moreover, personalized treatment parameters should improve treatment efficacy, and foster more widespread adoption of treatments that require personalized parameters. For example, subjects that were previously unable to take advantage of vagus nerve stimulation (“VNS”) due to preexisting obstructive sleep apnea (“OSA”) concerns, may be able to receive VNS with personalized treatment parameters developed using the stimulation systems described herein.

The present disclosure relates to improvements to functional sensing for implantable stimulation systems, such as, e.g., IPGs, and the present disclosure further relates to methods for energy-efficient cooperation between implanted sensors and external sensing devices.

In some aspects, one or more external sensors may be incorporated into a patient remote. In some aspects, external sensor(s) may be incorporated into a consumer electronic device, such as a smartphone. In some aspects, the external sensors may be incorporated into a wearable device, a portable device, or a stationary device (or any combination thereof).

In some aspects, wearable sensing devices may comprise, e.g., a smart watch, or a fingertip sensor such as a pulse oximeter. In some aspects, the wearable sensing device may be configured to emulate a piece of jewelry, such as, e.g., a ring, an earring, a bracelet, or a necklace.

In some aspects, the stimulation system may comprise one or more implantable stimulation devices (e.g., an IPG). In other aspects, the implantable stimulation device may comprise an implantable pump (e.g., an implantable opioid pump or an insulin pump).

In some aspects, the stimulation system may comprise one or more implantable stimulation devices, and each may include any one or a combination of the following implanted sensors: an accelerometer, an IMU, a magnetometer, a gyroscope, an electrocardiogram (“ECG”), a blood oxygen saturation (“SpO₂”) sensor, a blood pressure sensor, a glucometer, or a drug concentration sensor. In other aspects, different or additional sensors may be used.

In some aspects, the external or wearable device may include any one or a combination of the following sensors: an accelerometer, an IMU, a magnetometer, a gyroscope, an ECG, an SpO₂ sensor, a blood pressure sensor, a glucometer, or a drug concentration sensor. In other aspects, different or additional sensors may be used.

In another aspect, combination of the data from IMU and external sensor(s) included in the electronic device, may be used to detect the heart rate. Both IMU and external device may be configured to detect, for example, heart rate, thus offering the opportunity to compare the accuracy of the two sensors and notify the patient of a potential sensing error. If one sensor is susceptible to drift or other errors and needs periodic recalibration, the secondary sensor may provide a way to recalibrate. For example, implantable devices can rotate or flip. Using an external sensor that senses the beginning and end of inspiration may be used to detect flipping of the implant by signal inversion and detect rotation of the implant by change of the signal magnitude on each of the axes. Another benefit of having both an IMU and external sensor to sense heart rate is in closed loop stimulation in which a stimulator may have an algorithm to switch between sensing sources based on the quality of sensing signal on each and provide a more accurate stimulation therapy.

In some aspects, the implanted stimulation device and the external (or wearable) sensing device may both include one or more of the same type of sensor. Put another way, sensors within the external (or wearable) sensing device may, in some aspects, provide a redundant means for collecting desired sensory data.

In some aspects, one or more redundant external sensors may be configured to function in a cooperative and/or simultaneous manner with one or more implanted sensors to improve fidelity of a desired sensory data stream.

In some aspects, the one or more implanted sensors and the one or more redundant external sensors may be configured to function in an alternating manner, wherein only one of the one or more implanted sensors, and the one or more external sensors, are utilized at a time. Thus, the external (or wearable) sensors may reduce the energy burden upon an implantable medical device.

In some aspects, the one or more external (optionally, wearable) sensors may be configured to communicate with an implantable medical device via wireless connection. In some aspects, Bluetooth communication between one or more of the external sensors and the IMD may be used, e.g., to collect sensor data and/or to control the external sensor(s).

In some aspects, a stimulation system incorporating at least some of the aforementioned elements may be used for stimulation of a peripheral nerve, such as the vagus nerve, for the treatment of epilepsy. In other applications, such stimulation systems could be used for the treatment of, e.g., OSA, bladder dysfunction, chronic pain, or any other application treatable using electrical stimulation of one or more nerves, tissue, and/or organs.

In one general aspect, the disclosure provides a stimulation system, comprising an implantable stimulator configured to (a) deliver electrical stimulation to a nerve of a subject; and/or (b) deliver an active agent to one or more cells, tissues, or organs of the subject, and an implantable controller, configured to control the implantable stimulator; an electronic device, configured to wirelessly communicate with the implantable controller, and to set or control one or more parameters of the delivery of the electrical stimulation or active agent by the implantable stimulator; one or more implantable sensors, each configured to detect a signal indicative of a level of at least one biometric parameter of the subject, and to transmit the signal to the electronic device; one or more external sensors, each configured to detect a signal indicative of a level of at least one biometric parameter of the subject, and to transmit the signal to the electronic device; wherein at least one of the one or more external sensors is integrated into, affixed to, or placed on, the electronic device.

In some aspects, the electronic device comprises: a) a wearable device, optionally a watch, a ring, a bracelet, a band, a necklace, or an earring; b) a stationary device, optionally configured to be operated when the stationary device is placed on a surface (e.g., a bedside device); or c) a portable device, optionally comprising an external housing configured to be held or carried by the subject (e.g., a handheld dedicated controller or smart phone).

In some aspects, the implantable stimulator comprises: a) an implantable pulse generator (IPG) configured to stimulate a vagus nerve, a hypoglossal nerve, a sacral nerve, one or more pelvic parasympathetic nerves, one or more lumbar sympathetic nerves, one or more pudendal nerves, or one or more peripheral nerves, of the subject; or b) a pump configured to deliver insulin, baclofen, ziconotide, clonidine, bupivacaine, or an opioid to a bloodstream, organ, tissue, or any other body cavity or region of a body of the subject.

In some aspects, the electronic device comprises a smart watch.

In some aspects, the one or more implantable sensors comprise: an accelerometer, an inertial measurement unit (“IMU”), a magnetometer, a gyroscope, an electrocardiogram (“ECG”) sensor, a blood oxygen saturation (“SpO₂”) sensor, a blood pressure sensor, a glucometer, and/or a drug concentration sensor.

In some aspects, the one or more external sensors comprise: an accelerometer, an IMU, a magnetometer, a gyroscope, an ECG sensor, an SpO₂ sensor, a blood pressure, sensor, a glucometer, or a drug concentration sensor.

In some aspects, one or more of the one or more external sensors are integrated into, affixed to, or placed on, a watch, a ring, a bracelet, a band, a necklace, an earring, or an article of clothing.

In some aspects, the electronic device is further configured to receive: a) manual input from the subject, via a graphical user interface (“GUI”) provided by the electronic device, wherein the input comprises one or more parameters for the delivery of the electrical stimulation or active agent; and/or b) input received from a remote server, comprising one or more parameters for the delivery of the electrical stimulation or active agent.

In some aspects, the electronic device is configured to set or control one or more parameters of the delivery of the electrical stimulation to a nerve of a subject by setting or adjusting one or more parameters of the electrical stimulation, wherein the parameters comprise a pulse frequency, width, amplitude, and/or duty cycle, of the electrical stimulation.

In some aspects, the electronic device is configured to set or control one or more parameters of the delivery of the active agent to one or more cells, tissues, or organs of the subject, by setting or adjusting one or more parameters, wherein the one or more parameters comprise activation of a pump, deactivation of the pump, a pumping duration, a pumping rate, a volume of active agent to deliver by the pump, and/or a timing schedule for pumping.

In some aspects, the electronic device is further configured to determine one or more biometric parameters of the subject, based on the signal received from the at least one implantable sensor and/or the signal received from the at least one external sensor; and to activate, terminate, titrate, or adjust the delivery of the electrical stimulation or active agent by the implantable stimulator, based on the determined one or more biometric parameters.

In some aspects, the electronic device is further configured to determine one or more biometric parameters of the subject, based on the signal received from the at least one implantable sensor and/or the signal received from the at least one external sensor; determine that the subject is experiencing, or is likely to experience, a medical condition or event, based on the determined one or more biometric parameters of the subject; and to activate, terminate, titrate, or adjust, the delivery of the electrical stimulation or active agent by the implantable stimulator in response to determining that the subject is experiencing, or is likely to experience, a medical condition or event.

In some aspects, the medical condition or event is tachycardia, an apnea or hypopnea event, a bradycardia event, a potential opioid overdose, or a hypoglycemia event

In some aspects, the implantable stimulator comprising a housing and wherein the one or more implantable sensors include one or more sensors integrated into, affixed to, or placed on the housing; and wherein the implantable stimulator is configured to turn off one or more of the one or more sensors integrated into, affixed to, or placed on the housing in response to (a) a command transmitted by the electronic device, (b) determining that a wireless communication link has been established with the electronic device, or (c) determining that the electronic device is within a preset distance of or in proximity to the implantable stimulator

In some aspects, the system is configured to allow recalibration of the one or more implantable sensors using sensor data obtained from the one or more external sensors.

In some aspects, the implantable controller is further configured to measure or receive a signal quality parameter for each of the one or more internal sensors and the one or more external sensors; select a subset of sensors, comprising at least one of the one or more internal sensors and/or the one or more external sensors, based on the measured or received signal quality parameter(s); deliver the electrical stimulation and/or active agent based on sensor data obtained from the subset of sensors.

In another general aspect, the disclosure provides methods of treating any medical conditions or diseases described herein, using the foregoing systems, or any other systems set forth herein. For example, the disclosure provides a method of treating a subject, comprising: providing a medical device comprising an implantable stimulator configured to deliver (a) electrical stimulation to a nerve of a subject; and/or (b) an active agent to one or more cells, tissues, or organs of the subject, and an implantable controller, configured to control the implantable stimulator; receiving, by the implantable controller, one or more stimulation parameters from an electronic device via a wireless communication link; delivering the electrical stimulation or the active agent, by the implantable stimulator, based on the received one or more stimulation parameters, wherein the one or more stimulation parameters are based on (i) one or more biometric parameters of the subject determined using sensor data collected from one or more implantable sensors and one or more external sensors, (ii) user input from the subject, entered via a graphical user interface (“GUI”) provided by the electronic device, wherein the input comprises one or more parameters for the delivery of the electrical stimulation or active agent; and/or (iii) input received from a remote clinician, comprising one or more parameters for the delivery of the electrical stimulation or active agent.

In some aspects of the methods described herein, the electronic device comprises a) a wearable device, optionally a watch, a ring, a bracelet, a band, a necklace, or an earring; b) a stationary device, optionally configured to be operated when the stationary device is placed on a surface; or c) a portable device, optionally comprising an external housing configured to be held or carried by the subject.

In some aspects of the methods described herein, the the implantable stimulator comprises: a) an implantable pulse generator (“IPG”) configured to stimulate a vagus nerve, a hypoglossal nerve, a sacral nerve, one or more pelvic parasympathetic nerves, one or more lumbar sympathetic nerves, one or more pudendal nerves, or one or more peripheral nerves, of the subject; or b) a pump configured to deliver insulin, baclofen, ziconotide, clonidine, bupivacaine, or an opioid to a bloodstream, organ, tissue, or any other body cavity or region of a body of the subject.

In some aspects of the methods described herein, the one or more implantable sensors comprise an accelerometer, an inertial measurement unit (“IMU”), a magnetometer, a gyroscope, an electrocardiogram (“ECG”) sensor, a blood oxygen saturation (“SpO₂”) sensor, a blood pressure sensor, a glucometer, and/or a drug concentration sensor; and/or the one or more external sensors comprise: an accelerometer, an IMU, a magnetometer, a gyroscope, an ECG sensor, an SpO₂ sensor, a blood pressure, sensor, a glucometer, or a drug concentration sensor.

In some aspects of the methods described herein, the one or more stimulation parameters received from the electronic device cause the implantable controller to a) begin, terminate, or titrate the delivery of the electrical stimulation or active agent; b) set or adjust a pulse frequency, width, amplitude, and/or duty cycle, of the electrical stimulation; or c) activate or deactivate the pump, to pump for a preset duration of time, to pump until a preset volume of active agent has been delivered, to pump according to a timing schedule.

Ins some aspects of the methods described herein, the electronic device comprises a smart watch.

### BRIEF DESCRIPTION OF THE DRAWINGS

FIG. 1 is a block diagram showing an exemplary system according to the disclosure. As illustrated by this figure, the one or more implantable and/or external sensors, and/or the implantable controller, may communicate with an electronic device (e.g., a wearable device 110a, a stationary device 110b, a portable device 110c, or any other form factor).

FIG. 2 is a diagram showing the interaction between components of an exemplary system according to the disclosure.

FIG. 3 is a diagram showing the interaction between components of another exemplary system according to the disclosure.

FIG. 4 is a flowchart illustrating an exemplary method according to the disclosure. In this case, the method relates to a processing workflow for determining whether an external sensor should be selected (e.g., by an electronic device in the systems described herein) to replace an implantable sensor.

FIG. 5 is a flowchart illustrating an exemplary method according to the disclosure. In this case, the method relates to a processing workflow for determining whether a sensor (e.g., used by the systems described herein) requires recalibration.

FIG. 6 is a flowchart illustrating an exemplary method according to the disclosure. In this case, the method relates to a processing workflow for determining whether stimulation should be applied (or adjusted) as an intervention in response to one or more biometric parameters of a subject (e.g., determined by an electronic device using data collected from one or more implantable or external sensors).

FIG. 7 is a is a block diagram of various example system components, capable of being used along the lines as described in example implementations in accordance with aspects of the present disclosure.

### DETAILED DESCRIPTION

The detailed description set forth below in connection with the appended drawings is intended as a description of various configurations and is not intended to represent the only configurations in which the concepts described herein may be practiced. The detailed description includes specific details for the purpose of providing a thorough understanding of various concepts. However, it will be apparent to those skilled in the art that these concepts may be practiced without these specific details. In some instances, well known structures and components are shown in block diagram form in order to avoid obscuring such concepts.

Several aspects of exemplary aspects according to the present disclosure will now be presented with reference to various systems and methods. These systems and methods will be described in the following detailed description and illustrated in the accompanying drawings by various blocks, components, circuits, processes, algorithms, etc. (collectively referred to as “elements”). These elements may be implemented using electronic hardware, computer software, or any combination thereof. Whether such elements are implemented as hardware or software depends upon the particular application and design constraints imposed on the overall system.

By way of example, an element, or any portion of an element, or any combination of elements may be implemented as a “processing system” or “controller” that includes one or more processors. Examples of processors include microprocessors, microcontrollers, graphics processing units (“GPUs”), central processing units (“CPUs”), application processors, digital signal processors (“DSPs”), reduced instruction set computing (“RISC”) processors, systems on a chip (“SoC”), baseband processors, field programmable gate arrays (“FPGAs”), programmable logic devices (“PLDs”), application-specific integrated circuits (“ASICs”), state machines, gated logic, discrete hardware circuits, and other suitable hardware configured to perform the various functionality described throughout this disclosure. One or more processors in the processing system may execute software. Software shall be construed broadly to mean instructions, instruction sets, code, code segments, program code, programs, subprograms, software components, applications, software applications, software packages, routines, subroutines, objects, executables, threads of execution, procedures, functions, etc., whether referred to as software, firmware, middleware, microcode, hardware description language, or otherwise.

Accordingly, in one or more exemplary embodiments, the functions described may be implemented in hardware, software, or any combination thereof. If implemented in software, the functions may be stored on or encoded as one or more instructions or code on a computer-readable medium. Computer-readable media includes computer storage media. Storage media may be any available media that can be accessed by a computer. By way of example, and not limitation, such computer-readable media can comprise a random-access memory (“RAM”), a read-only memory (“ROM”), an electrically erasable programmable ROM (“EEPROM”), optical disk storage, magnetic disk storage, other magnetic storage devices, combinations of the aforementioned types of computer-readable media, or any other medium that can be used to store computer executable code in the form of instructions or data structures that can be accessed by a computer.

The present disclosure relates to improvements to functional sensing for implantable stimulation systems, such as, e.g., IPGs, and, in some aspects, to methods for energy-efficient cooperation between implanted sensors and external sensing devices.

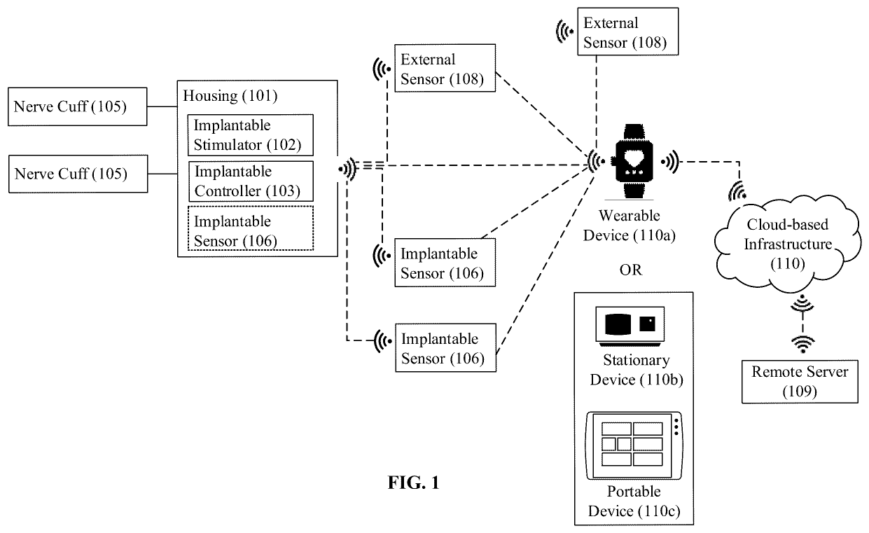

FIG. 1 is a block diagram of an exemplary system according to the present disclosure. This particular example illustrates a stimulation system comprising a housing 101, containing an implantable stimulator 102 (e.g., an IPG), an implantable controller 103 (e.g., comprising a processor, a memory, and software code that when executed controls the stimulation system or components thereof). The stimulation system further includes an implantable sensor 106 within the housing 101. Implantable sensors 106 may be integrated into an implanted stimulation system, placed on or connected to an implanted stimulation system, or be implanted separately (e.g., in a separate housing, configured for wireless or wired communication with the housing 101 of the implanted stimulation system. This diagram further illustrates two nerve cuffs 105 connected to the housing via leads. As explained herein, stimulation systems according to the disclosure may be used to deliver electrical stimulation to one or more nerves (e.g., the hypoglossal or vagus nerves) to treat OSA, seizures, or other medical conditions. It is envisioned that the wireless communication links shown in this figure may be potentially bidirectional or unidirectional.

In this example, the housing 101 is shown to be in wireless communication with multiple implantable sensors 106 and one external sensor 108. Wireless communication may be enabled by a Bluetooth, near-field communication (“NFC”), or other wireless communication protocol using a wireless modem integrated into the stimulation system. In this example, the wireless modem is envisioned as a component of the implantable controller 103 integrated into the housing 101 of the stimulation system. In other aspects, one or mor sensors may be communicatively linked to the implantable controller 103 via a physical connection (e.g., one or more implantable sensors 106 may be connected to the housing 101 and communicatively linked to the implantable controller 103 via electrical leads).

This figure further illustrates the use of an electronic device 110, in this case a wearable device 110a, in a smart watch form factor. The electronic device 110 is shown to be in wireless communication with two implantable sensors 106 and two external sensors 108, as well as with a cloud-based infrastructure 110 that provides a communication link to a remote server 109. The electronic device 110 may be configured to connect to a remote server 109 in order to obtain or transmit information (e.g., biometric parameters of the subject, or stimulation parameters to be used by the stimulation system). For example, the server may be operated by a medical professional (e.g., a clinician programmer) responsible for medical treatment of the subject. The medical professional may use the remote server to view and/or adjust one or more stimulation parameters (e.g., they may set or adjust a pulse frequency, width, amplitude, and/or duty cycle, of the electrical stimulation) or set conditions for when stimulation should be applied (e.g., one or more biometric parameter thresholds that trigger a therapeutic intervention, such as the activation of stimulation or an increase/decrease in stimulation).

In some aspects, the electronic device 110 may be configured to upload historical data to the remote server 109, such as historical biometric parameter levels of the subject, recorded medical events (e.g., onset or duration and/or timing of tachycardia, a seizure, or an apnea/hypopnea event). Such data may be used by a medical professional to monitor and/or evaluate the effect of stimulation parameters, so that therapy may be personalized for the subject.

In some aspects, the electronic device will comprise a controller capable of setting or modifying one or more parameters of the implanted stimulation system. It is envisioned that this functionality may allow the electronic device 110 to act as a master or primary controller of the implanted stimulation system (when available), with the implantable controller 103 serving as a secondary or backup controller to maintain operation (e.g., when the electronic device 110 is not in proximity or otherwise unavailable to the subject).

As illustrated by this figure, the electronic device 110 and the implantable controller 103 may each be in communication with one or more implantable sensors 106 and/or external sensors 108. In some aspects, both devices may maintain separate connections to one or more of the same sensors. However, it is envisioned that energy may be conserved by reducing the number of connections. For example, when the electronic device 110 is available (e.g., detected based on proximity or by the establishment of a communications link with the implantable controller 103), the implantable controller 103 may disable communication with one or more implantable sensors 106 or external sensors 108 in order to conserve the battery of the implanted stimulation system, with the electronic device 110 then taking over collection of sensor data. The electronic device 110 may establish a connection (or maintain a connection) with one or more of the implantable sensors 106 or external sensors 108 that were previously linked with the implantable controller 103. In other aspects, the electronic device 110 may use additional or alternative sensors as a replacement for one or more sensors disabled by the implantable controller 103. For example, the implantable controller 103 may disable an internal IMU used to detect the subject's respiration rate, and the electronic device 110 may obtain functionally equivalent data from an alternative external sensor 108. The electronic device 110 may use the sensor data collected from one or more of the implantable sensors 106 or external sensors 108 to determine one or more biometric parameters of the subject, and to determine whether stimulation is needed or if any stimulation parameters should be adjusted. When it is determined that stimulation is needed (of if stimulation parameters should be adjusted), the electronic device 110 may communicate any such instructions or commands to the implantable controller 103 in order to trigger stimulation according to the selected parameters.

The electronic device 110 will typically have a graphical user interface (“GUI”) allowing a subject to view and/or modify one or more parameters of the stimulation system, or to access other information about the stimulation system. For example, the electronic device 110 may provide a GUI that allows a subject to turn off or turn on stimulation, to set a schedule for the system (e.g., a system for treating OSA would typically be enabled during the time range when the subject is asleep). The GUI may also allow the user to immediately disable stimulation (e.g., a system for treating OSA might inadvertently be triggered by a subject laying in a prone position while awake).

In some aspects, the implantable controller 103 may be configured to switch to a low power mode when the electronic device 110 is available. In this mode, the implantable controller 103 may disable one or more implantable sensors 106 or external sensors 108, and/or may reduce a sensor polling rate or frequency of communication with one or more implantable sensors 106 or external sensors 108. The system may allow a user to manually activate this low power mode (e.g., using a GUI of the electronic device 110). In some aspects, the implantable controller 103 may be switched back to a fully active mode based upon a present condition (e.g., the termination of a wireless link with the electronic device 110, a manual command input by the subject, or inability to detect the electronic device 110 within a given amount of time). In the fully active mode, the implantable controller 103 may resume control over stimulation and may optionally cease stimulation or any previous stimulation parameters. In some aspects, the implantable controller 103 may be configured to poll all available sensors, or a predetermined set of sensors, upon entering fully active mode. For example, the implantable controller 103 may be configured to terminate all previous stimulation parameters upon entry into fully active mode, to poll all available sensors, and to proceed to determine one or more biometric parameters of the subject and whether intervention is needed. As such, the implantable controller 103 may function as a backup controller capable of taking over the sensor monitoring and stimulation control features provided by the electronic device 110. This backup functionality may be useful, e.g., in cases where the electronic device 110 becomes inoperable suddenly due to battery depletion or some other cause.

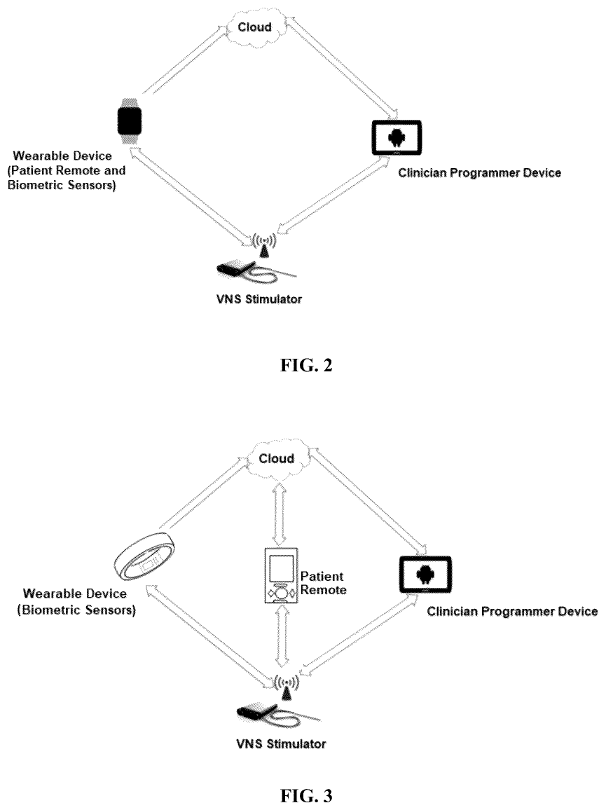

FIG. 2 is a diagram illustrating a stimulation system that includes an implantable stimulator and an electronic device 110 (more specifically, examples of a wearable device 110a in the form factor of a smart watch) used for biometric sensing and for controlling the implanted stimulator. The electronic device 110 may include Bluetooth to connect to the implantable stimulator and provide a simple user interface for user to control stimulation parameters (e.g., the parameters allowed to be controlled by a patient). The electronic device 110 may be configured to function as a patient remote and for biometric sensing (e.g., heart rate, ECG, SpO₂, blood pressure etc.). The electronic device 110 may also have cellular and/or wi-fi connectivity to communicate with a server (e.g., a cloud-based server) for data sync and/or remote programming in a cloud-based environment. For example, data collected from electronic device(s) 110 and/or the implantable stimulator may be uploaded to a cloud server for remote data processing. Data can be uploaded using cellular or wi-fi connectivity. In certain conditions, specific events e.g. SpO₂ level could be transmitted to the implantable stimulator and the implantable stimulator can change the stimulation accordingly.

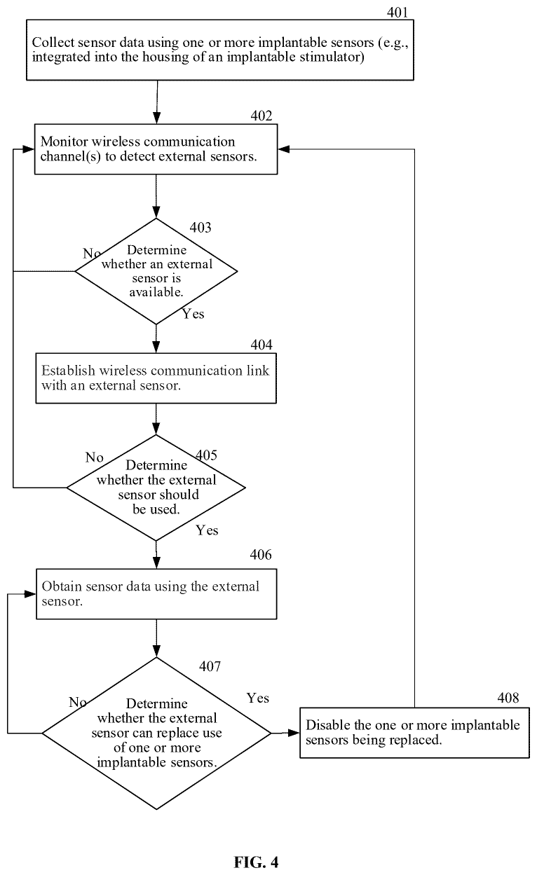

In another embodiment shown in FIG. 3, a first electronic device 110 (e.g., a ring or other wearable device 110a) is used for biometric sensing (heart rate sensor, oxygen sensor, blood pressure sensor, etc.) and a second electronic device 110 is used as a primary controller of the stimulation system (e.g., the handheld patient remote, which comprises a portable device 110c). It should be understood that any of the systems described herein may incorporate a plurality of electronic devices 110 (e.g., one or more wearable devices 110a, stationary devices 110b, or portable devices 110c). Control and sensing functionality may be divided among the plurality of electronic devices 110 in any manner (e.g., one electronic device 110 may serve as a primary controller, with the others as backup or secondary controllers).

In some aspects, the stimulation system may utilize a portable device 110c (e.g., a handheld smart phone or dedicated control unit) configured to communicate with and control an implantable stimulator. In some aspects, the patient's biometric data is collected using sensors integrated into or communicatively linked to the portable device 110c, or to a different electronic device 110, such as a wearable device 110a (e.g., a smart watch) or a stationary device 110b (e.g., a bedside unit) configured to communicate with the portable device 110c. As noted above, the control and sensing functions described herein may be performed by any electronic device 110 described herein, or by a combination of such devices (e.g., where each devices performs some or all of the control and/or sensing functionality). In some aspects, a stimulation system may comprise a plurality of electronic devices 110, each assigned a priority rank, where the control and/or sensing functionality is performed by the highest ranking electronic device 110 detected in proximity to the subject or the implantable controller 103 (or based on any other criteria). For example, a stimulation system may prioritize use of a wearable device 110a when a stationary device 110b is not detected or where a wireless signal emitted by the stationary device 110b is below a preset threshold, and to switch control to the stationary device 110b when that unit is detected or where a wireless signal emitted by the stationary device 110b is above a preset threshold. The same handoff process may occur with use of a portable device 110c. In other aspects, control may be assumed by the electronic device 110 that has the highest battery level, greatest processing capabilities, strongest wireless signal, or any other rule. Alternatively, the user or a clinician programmer may manually set or select an electronic device 110 as the primary controller (or set any other prioritization rules or assignments).

External sensing devices such as smart watches often detect heart rate using PPG (photoplethysmography). This signal is typically less sensitive to patient motion than is an implanted IMU, because the IMU senses motion by design. Therefore, using an external PPG will provide a less motion-sensitive detection of heart rate and respiration, which can be recovered from the heart rate signal. It is also more convenient to take a watch off and recharge a smartwatch than it is to don an implant charger to recharge one's implantable device.

External devices also have some drawbacks. They are not implanted and thus are not guaranteed to be worn at all times. It is commonplace to recharge smart watches at night, especially for some individuals that cannot tolerate wearing jewelry, watches, or other devices at night. Thus, it is convenient to use an IMU to detect the required physiologic parameters when the patient sleeps, bathes, or is not wearing the external device for any other reason.

Whenever a suitable external accessory is available and connected to the IPG, the IPG may be configured to cease its sensing capabilities once sensing data is available via the external accessory. For example, when heart rate monitoring data is available via a smart watch, then the IPG may stop utilizing its IMU to sense heart rate variability. FIG. 4 is a flowchart providing a general example of this type of functionality. In this case, the method relates to a processing workflow for determining whether an external sensor should be selected (e.g., by a electronic device 110 in the systems described herein) to replace an implantable sensor. In this example, it is envisioned that an implantable stimulation system may collect sensor data using one or more implantable sensors 106 (e.g., integrated into the housing 101 of an implantable stimulator 102. The system may be configured to monitor wireless communication channels to detect external sensors 108. When one is detected, the system may establish a communications link with the external sensor and determine whether it should be used. This determination may be made, e.g., based on a signal quality parameter, or logic regarding the selection of sensors. For example, the implantable controller 103 or the electronic device 110 may be configured to select and use sensors having a signal quality above a predetermined threshold, or may be configured to select only certain types of sensors, or a minimum/maximum number of sensors of a certain type of class. The system may also choose sensors based on manual user input, input from a clinician programmer (e.g., provided by a remote server 109 operated by a clinician programmer), a schedule (e.g., one or more sensors may be selected for use at different times of day). In some aspects, the system may determine whether a sensor should be used by comparing the accuracy of its signal compared to sensor data collected from one or more other sensors (e.g., a comparison may show that sensor data from the new sensor is out of sync with data from a plurality of other sensors associated with he same biometric parameter).

If it is determined that a sensor should be used, the system may then utilize that sensor to collect sensor data. At that stage, the system may then be optionally configured to determine whether the new sensor can function as a replacement for another sensor that is currently in use. For example, the system may have access to multiple sensors capable of obtaining data indicative of the respiratory rate of a subject. In this situation, multiple sensors may be used simultaneously (e.g., to improve accuracy and/or to detect when any individual sensor falls out of sync due to movement or other artifacts), or a single or reduced subset of sensors may be used (e.g., to conserve battery life). The system may follow predetermined logic when making this determination. For example, the system may be configured to select a replacement sensor capable of providing a signal indicative of a biometric parameter currently being monitored, when the new sensor provides energy efficiency, has a longer battery life, is external/internal, has a higher signal quality parameter, is identified as having higher accuracy than the current sensor, etc. It is envisioned that any parameters disclosed herein may be used to determine whether a sensor can replace use of another sensor.

When external accessories are not available or sensing data is not reliable, the IPG maybe configured to turn its internal sensing ON (e.g., by utilizing or activating the implantable controller 103 in order to collect sensor data from implantable sensors 106 or external sensors 108).

The algorithm can be applied to sensing but not limited to sensing only i.e. similar approach can be applied to stimulation decision making as well e.g. smart watch detects the ictal tachycardia and sends a command to IPG to increase the stimulation duty cycle.

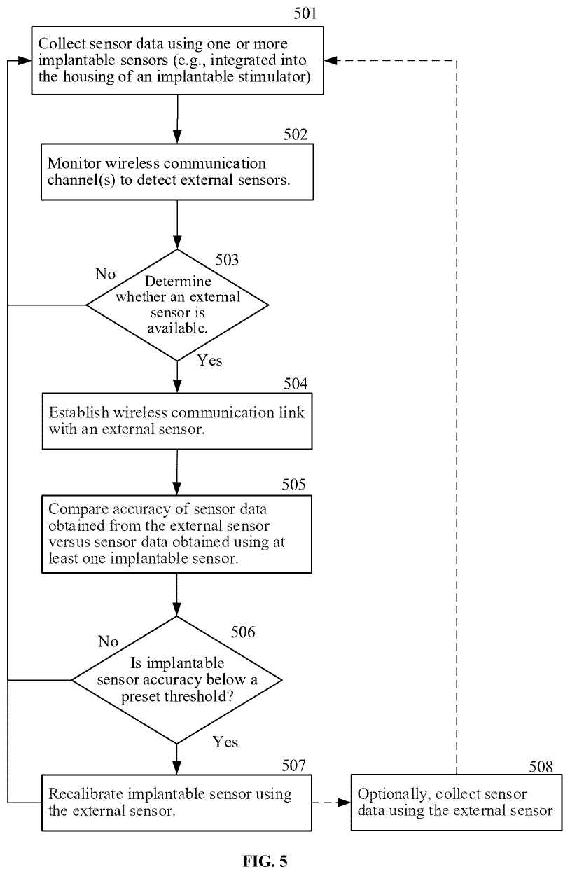

Having two devices to sense any physiological parameter also provides the opportunity to compare the accuracy of the two sensors and notify the patient of a potential sensing error. If one sensor is susceptible to drift or other errors and needs periodic recalibration, the secondary sensor can provide a way to recalibrate. For example, implantable devices can rotate or flip. Using an external sensor that senses the beginning and end of inspiration can be use to detect flipping of the implant by signal inversion and detect rotation of the implant by change of the signal magnitude on each of the axes. FIG. 5 is a flowchart illustrating an exemplary method according to the disclosure for detecting sensor drift and triggering recalibration. In this case, the process begins with the collection of sensor data from one or more sensors (e.g., by the implantable controller 103 or electronic device 110 of a stimulation system described herein). The system may be configured to monitor wireless communication channels to detect one or more additional sensors are available (in this case, detection of an external sensor is shown). After establishing wireless communication link with the new sensor, the system may then compare the accuracy of the sensor data obtained from the new sensor versus sensor data obtained using at least one other sensor (in this case, an implantable sensor is shown). If the accuracy of the other sensor is found to be less than the accuracy of the new sensor, the system may then proceed to recalibrate the other sensor using the sensor as a baseline. In this case, the comparison included a threshold level of inaccuracy as the trigger for recalibration. During (or after) the recalibration, the system may proceed to utilize the new sensor as a replacement for the old sensor.

This figure illustrates one non-limiting example of a method for detecting sensor drift and performing a recalibration. Other methods may proceed using a different workflow and/or parameters. For example, a system may be configured to determine whether a sensor is inaccurate by periodically comparing the sensor data obtained from that sensor against a baseline sensor, or a plurality of sensors, capable of detecting a signal indicative of the same biometric parameter (or a different biometric parameter that can serve as a proxy for the biometric parameter at issue). A sensor may be found to be inaccurate of the determination of a biometric parameter based on that sensor's signal differs from the same determination made using sensor data obtained from the selected baseline sensor, or from an average or median value for a biometric parameter obtained using a plurality of sensors. As with the workflow shown in FIG. 5, a threshold may be used (e.g., a deviation of 1, 2, 3, 4, 5, 6, 7, 8, 9, 10, 15, or 20%, or a deviation within a range defined by any pair of integers between 1 and 100%)

Another benefit of having two devices to sense is in closed loop stimulation where IPG can have an algorithm to switch between sensing sources based on the quality of sensing signal on each and provide more accurate therapy.

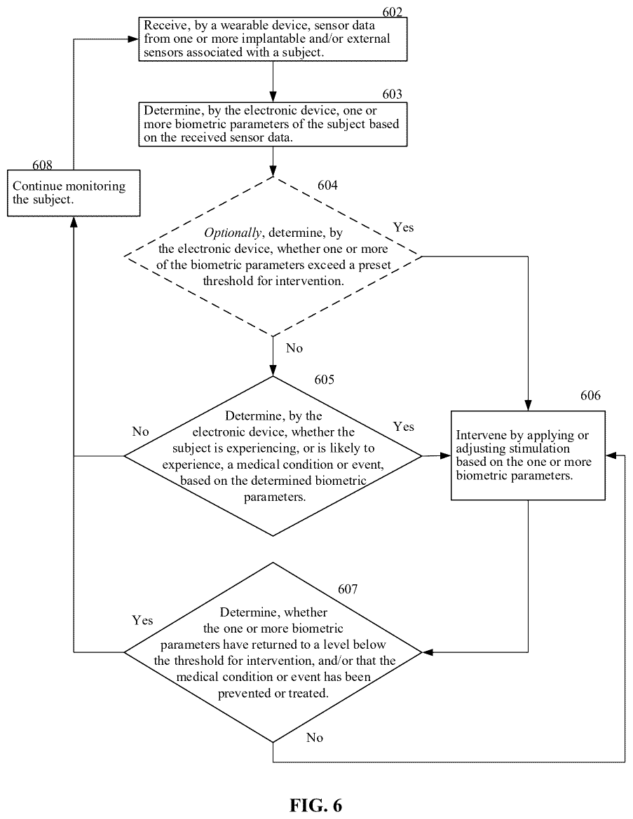

FIG. 6 is a flowchart illustrating an exemplary method according to the disclosure. In this case, the method relates to a processing workflow for determining whether stimulation should be applied (or adjusted) as an intervention in response to one or more biometric parameters of a subject (e.g., determined by an electronic device 110 using data collected from one or more implantable or external sensors). In this example a stimulation system includes an electronic device 110 capable of receiving sensor data from one or more implantable sensors 106 and/or external sensors 108. The electronic device 110 may utilize this sensor data to determine one or more biometric parameters of the subject (e.g., heart rate, respiration rate, respiration cycle, the subject's temperature at one or more physiological locations, the subject's position). The electronic device 110 may then determine whether one or more of the determined biometric parameters exceeds a preset threshold (it is envisioned that “exceeds” in this context may refer to a level that is above or below a normal or safe limit, and is not limited strictly to levels that are simply numerically higher than a threshold). If so, the electronic device 110 may trigger intervention by communicating with the implantable controller 103 of the stimulation system and triggering stimulation (or adjusting one or more stimulation parameters). This type of intervention is optional. In other aspects, the system may not be configured to intervene based on the determination of a biometric parameters threshold being exceeded and may instead may be configured to intervene following a more complex determination—e.g., the determination that a subject is experiencing, or is likely to experience, a medical condition or event. This determination may take into account one or more biometric parameters and optionally thresholds regarding the same, in addition to other data. For example, the determination may not be limited to biometric parameters at a given timepoint and may instead take into account historical data (e.g., a determination that the subject is experiencing an apnea/hypopnea event may be based on the subject's respiratory rate and/or respiratory cycle, and also take into account the time of day, historical data obtained from the subject recorded during an apnea/hypopnea event, sleep study data, or other information). Upon determining that an intervention is appropriate, the system may then proceed to intervene by triggering stimulation or adjustment of stimulation parameters. As illustrated by this figure, stimulation and monitoring may continue until the subject's biometric parameters have returned to a normal level (e.g., determined using one or more thresholds), at which time the system may return to the initial stage of the processing workflow.

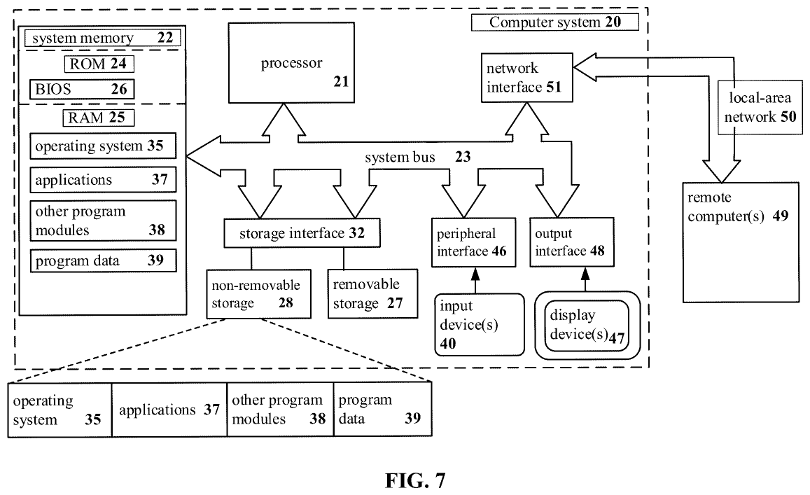

FIG. 7 is a is a block diagram of various exemplary system components, capable of being used along the lines as described in example implementations in accordance with aspects of the present disclosure.

Aspects of the present disclosure may be implemented using hardware, software, or a combination thereof and may be implemented in one or more computer systems or other processing systems. In an aspect of the present disclosure, features are directed toward one or more computer systems capable of carrying out the functionality described herein. FIG. 6 is a block diagram illustrating an example of a computer system 20 which may be used to implement aspects of the systems and methods described herein. The computer system 20 can be in the form of multiple computing devices, or in the form of a single computing device, for example, a desktop computer, a notebook computer, a laptop computer, a mobile computing device, a smart phone, a tablet computer, a server, a mainframe, an embedded device, and other forms of computing devices.

As shown, the computer system 20 includes a central processing unit (CPU) 21, a system memory 22, and a system bus 23 connecting the various system components, including the memory associated with the central processing unit 21. The system bus 23 may comprise a bus memory or bus memory controller, a peripheral bus, and a local bus that is able to interact with any other bus architecture. Examples of the buses may include PCI, ISA, PCI-Express, HyperTransport™, InfiniBand™, Serial ATA, I2C, and other suitable interconnects. The central processing unit 21 (also referred to as a processor) can include a single or multiple sets of processors having single or multiple cores. The processor 21 may execute one or more computer-executable code implementing the techniques of the present disclosure. For example, any of commands/steps discussed in this specification, or shown in the accompanying drawings, may be performed by processor 21. The system memory 22 may be any memory for storing data used herein and/or computer programs that are executable by the processor 21. The system memory 22 may include volatile memory such as a random access memory (RAM) 25 and non-volatile memory such as a read only memory (ROM) 24, flash memory, etc., or any combination thereof. The basic input/output system (BIOS) 26 may store the basic procedures for transfer of information between elements of the computer system 20, such as those at the time of loading the operating system with the use of the ROM 24.

The computer system 20 may include one or more storage devices such as one or more removable storage devices 27, one or more non-removable storage devices 28, or a combination thereof. The one or more removable storage devices 27 and non-removable storage devices 28 are connected to the system bus 23 via a storage interface 32. In an aspect, the storage devices and the corresponding computer-readable storage media are power-independent modules for the storage of computer instructions, data structures, program modules, and other data of the computer system 20. The system memory 22, removable storage devices 27, and non-removable storage devices 28 may use a variety of computer-readable storage media. Examples of computer-readable storage media include machine memory such as cache, SRAM, DRAM, zero capacitor RAM, twin transistor RAM, eDRAM, EDO RAM, DDR RAM, EEPROM, NRAM, RRAM, SONOS, PRAM; flash memory or other memory technology such as in solid state drives (SSDs) or flash drives; magnetic cassettes, magnetic tape, and magnetic disk storage such as in hard disk drives or floppy disks; optical storage such as in compact disks (CD-ROM) or digital versatile disks (DVDs); and any other medium which may be used to store the desired data and which can be accessed by the computer system 20.

The system memory 22, removable storage devices 27, and non-removable storage devices 28 of the computer system 20 may be used to store an operating system 35, additional program applications 37, other program modules 38, and program data 39. The computer system 20 may include a peripheral interface 46 for communicating data from input devices 40, such as a keyboard, mouse, stylus, game controller, voice input device, touch input device, or other peripheral devices, such as a printer or scanner via one or more I/O ports, such as a serial port, a parallel port, a universal serial bus (USB), or other peripheral interface. A display device 47 such as one or more monitors, projectors, or integrated display, may also be connected to the system bus 23 across an output interface 48, such as a video adapter. In addition to the display devices 47, the computer system 20 may be equipped with other peripheral output devices (not shown), such as loudspeakers and other audiovisual devices.

The computer system 20 may operate in a network environment, using a network connection to one or more remote computers 49. The remote computer (or computers) 49 may be local computer workstations or servers comprising most or all of the aforementioned elements in describing the nature of a computer system 20. Other devices may also be present in the computer network, such as, but not limited to, routers, network stations, peer devices or other network nodes. The computer system 20 may include one or more network interfaces 51 or network adapters for communicating with the remote computers 49 via one or more networks such as a local-area computer network (LAN) 50, a wide-area computer network (WAN), an intranet, and the Internet. Examples of the network interface 51 may include an Ethernet interface, a Frame Relay interface, SONET interface, and wireless interfaces.

Aspects of the present disclosure may be a system, a method, and/or a computer program product. The computer program product may include a computer readable storage medium (or media) having computer readable program instructions thereon for causing a processor to carry out aspects of the present disclosure.

The computer readable storage medium can be a tangible device that can retain and store program code in the form of instructions or data structures that can be accessed by a processor of a computing device, such as the computing system 20. The computer readable storage medium may be an electronic storage device, a magnetic storage device, an optical storage device, an electromagnetic storage device, a semiconductor storage device, or any suitable combination thereof. By way of example, such computer-readable storage medium can comprise a random access memory (RAM), a read-only memory (ROM), EEPROM, a portable compact disc read-only memory (CD-ROM), a digital versatile disk (DVD), flash memory, a hard disk, a portable computer diskette, a memory stick, a floppy disk, or even a mechanically encoded device such as punch-cards or raised structures in a groove having instructions recorded thereon. As used herein, a computer readable storage medium is not to be construed as being transitory signals per se, such as radio waves or other freely propagating electromagnetic waves, electromagnetic waves propagating through a waveguide or transmission media, or electrical signals transmitted through a wire.

Computer readable program instructions described herein can be downloaded to respective computing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network interface in each computing device receives computer readable program instructions from the network and forwards the computer readable program instructions for storage in a computer readable storage medium within the respective computing device.

Computer readable program instructions for carrying out operations of the present disclosure may be assembly instructions, instruction-set-architecture (ISA) instructions, machine instructions, machine dependent instructions, microcode, firmware instructions, state-setting data, or either source code or object code written in any combination of one or more programming languages, including an object-oriented programming language, and conventional procedural programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a LAN or WAN, or the connection may be made to an external computer (for example, through the Internet). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present disclosure.

In various aspects, the systems and methods described in the present disclosure can be addressed in terms of modules. The term “module” as used herein refers to a real-world device, component, or arrangement of components implemented using hardware, such as by an application specific integrated circuit (ASIC) or FPGA, for example, or as a combination of hardware and software, such as by a microprocessor system and a set of instructions to implement the module's functionality, which (while being executed) transform the microprocessor system into a special-purpose device. A module may also be implemented as a combination of the two, with certain functions facilitated by hardware alone, and other functions facilitated by a combination of hardware and software. In certain implementations, at least a portion, and in some cases, all, of a module may be executed on the processor of a computer system. Accordingly, each module may be realized in a variety of suitable configurations, and should not be limited to any particular implementation exemplified herein.

In the interest of clarity, not all of the routine features of the aspects are disclosed herein. It would be appreciated that in the development of any actual implementation of the present disclosure, numerous implementation-specific decisions must be made in order to achieve the developer's specific goals, and these specific goals will vary for different implementations and different developers. It is understood that such a development effort might be complex and time-consuming, but would nevertheless be a routine undertaking of engineering for those of ordinary skill in the art, having the benefit of this disclosure.

Furthermore, it is to be understood that the phraseology or terminology used herein is for the purpose of description and not of restriction, such that the terminology or phraseology of the present specification is to be interpreted by the skilled in the art in light of the teachings and guidance presented herein, in combination with the knowledge of those skilled in the relevant art(s). Moreover, it is not intended for any term in the specification or claims to be ascribed an uncommon or special meaning unless explicitly set forth as such.

The various aspects disclosed herein encompass present and future known equivalents to the known modules referred to herein by way of illustration. Moreover, while aspects and applications have been shown and described, it would be apparent to those skilled in the art having the benefit of this disclosure that many more modifications than mentioned above are possible without departing from the inventive concepts disclosed herein.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular compound, composition, article, apparatus, methodology, protocol, and/or reagent, etc., described herein, unless expressly stated as such. In addition, those of ordinary skill in the art will recognize that certain changes, modifications, permutations, alterations, additions, subtractions and sub-combinations thereof can be made in accordance with the teachings herein without departing from the spirit of the present specification. It is therefore intended that the following appended claims and claims hereafter introduced are interpreted to include all such changes, modifications, permutations, alterations, additions, subtractions and sub-combinations as are within their true spirit and scope.

Certain aspects of the present disclosure are described herein, including the best mode known to the inventors for carrying out the disclosure. Of course, variations on these described embodiments will become apparent to those of ordinary skill in the art upon reading the foregoing description. The inventor expects skilled artisans to employ such variations as appropriate, and the inventors intend for the present disclosure to be practiced otherwise than specifically described herein. Accordingly, this disclosure includes all modifications and equivalents of the subject matter recited in the claims appended hereto as permitted by applicable law. Moreover, any combination of the above-described embodiments in all possible variations thereof is encompassed by the disclosure unless otherwise indicated herein or otherwise clearly contradicted by context.

Groupings of alternative embodiments, elements, or steps of the present disclosure are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques.

Use of the terms “may” or “can” in reference to an embodiment or aspect of an embodiment also carries with it the alternative meaning of “may not” or “cannot.” As such, if the present specification discloses that an embodiment or an aspect of an embodiment may be or can be included as part of the inventive subject matter, then the negative limitation or exclusionary proviso is also explicitly meant, meaning that an embodiment or an aspect of an embodiment may not be or cannot be included as part of the inventive subject matter. In a similar manner, use of the term “optionally” in reference to an embodiment or aspect of an embodiment means that such embodiment or aspect of the embodiment may be included as part of the inventive subject matter or may not be included as part of the inventive subject matter. Whether such a negative limitation or exclusionary proviso applies will be based on whether the negative limitation or exclusionary proviso is recited in the claimed subject matter.

Notwithstanding that the numerical ranges and values setting forth the broad scope of the disclosure are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

The terms “a,” “an,” “the” and similar references used in the context of describing aspects of the present disclosure (especially in the context of the following claims) are to be construed to cover both the singular and the plural, unless otherwise indicated herein or clearly contradicted by context. Further, ordinal indicators-such as “first,” “second,” “third,” etc.—for identified elements are used to distinguish between the elements, and do not indicate or imply a required or limited number of such elements, and do not indicate a particular position or order of such elements unless otherwise specifically stated. All methods described herein can be performed in any suitable order unless otherwise indicated herein or otherwise clearly contradicted by context. The use of any and all examples, or “exemplary” language (e.g., “such as”) provided herein is intended merely to better illuminate the present disclosure and does not pose a limitation on the scope of the invention otherwise claimed. No language in the present specification should be construed as indicating any non-claimed element essential to the practice of the invention.

When used in the claims, whether as filed or added per amendment, the open-ended transitional term “comprising” (and equivalent open-ended transitional phrases thereof like including, containing and having) encompasses all the expressly recited elements, limitations, steps and/or features alone or in combination with unrecited subject matter; the named elements, limitations and/or features are essential, but other unnamed elements, limitations and/or features may be added and still form a construct within the scope of the claim. Specific embodiments disclosed herein may be further limited in the claims using the closed-ended transitional phrases “consisting of” or “consisting essentially of” in lieu of or as an amended for “comprising.” When used in the claims, whether as filed or added per amendment, the closed-ended transitional phrase “consisting of” excludes any element, limitation, step, or feature not expressly recited in the claims. The closed-ended transitional phrase “consisting essentially of” limits the scope of a claim to the expressly recited elements, limitations, steps and/or features and any other elements, limitations, steps and/or features that do not materially affect the basic and novel characteristic(s) of the claimed subject matter. Thus, the meaning of the open-ended transitional phrase “comprising” is being defined as encompassing all the specifically recited elements, limitations, steps and/or features as well as any optional, additional unspecified ones. The meaning of the closed-ended transitional phrase “consisting of” is being defined as only including those elements, limitations, steps and/or features specifically recited in the claim whereas the meaning of the closed-ended transitional phrase “consisting essentially of” is being defined as only including those elements, limitations, steps and/or features specifically recited in the claim and those elements, limitations, steps and/or features that do not materially affect the basic and novel characteristic(s) of the claimed subject matter. Therefore, the open-ended transitional phrase “comprising” (and equivalent open-ended transitional phrases thereof) includes within its meaning, as a limiting case, claimed subject matter specified by the closed-ended transitional phrases “consisting of” or “consisting essentially of.” As such embodiments described herein or so claimed with the phrase “comprising” are expressly or inherently unambiguously described, enabled and supported herein for the phrases “consisting essentially of” and “consisting of.”

All patents, patent publications, and other publications referenced and identified in the present specification are individually and expressly incorporated herein by reference in their entirety for the purpose of describing and disclosing, for example, the compositions and methodologies described in such publications that might be used in connection with the present disclosure. These publications are provided solely for their disclosure prior to the filing date of the present application. Nothing in this regard should be construed as an admission that the inventors are not entitled to antedate such disclosure by virtue of prior disclosure or for any other reason. All statements as to the date or representation as to the contents of these documents is based on the information available to the applicants and does not constitute any admission as to the correctness of the dates or contents of these documents.

Lastly, the terminology used herein is for the purpose of describing particular embodiments only, and is not intended to limit the scope of the present invention, which is defined solely by the claims. Accordingly, the present invention is not limited to that precisely as shown and described.

1. A stimulation system, comprising:
an implantable stimulator configured to (a) deliver electrical stimulation to a nerve of a subject; and/or (b) deliver an active agent to one or more cells, tissues, or organs of the subject, and
an implantable controller, configured to control the implantable stimulator;
an electronic device, configured to wirelessly communicate with the implantable controller, and to set or control one or more parameters of the delivery of the electrical stimulation or active agent by the implantable stimulator;
one or more implantable sensors, each configured to detect a signal indicative of a level of at least one biometric parameter of the subject, and to transmit the signal to the electronic device;
one or more external sensors, each configured to detect a signal indicative of a level of at least one biometric parameter of the subject, and to transmit the signal to the electronic device;
wherein at least one of the one or more external sensors is integrated into, affixed to, or placed on, the electronic device.

2. The stimulation system of claim 1, wherein the electronic device comprises:
a) a wearable device, optionally a watch, a ring, a bracelet, a band, a necklace, or an earring;
b) a stationary device, optionally configured to be operated when the stationary device is placed on a surface; or
c) a portable device, optionally comprising an external housing configured to be held or carried by the subject.

3. The stimulation system of claim 1, wherein the implantable stimulator comprises:
a) an implantable pulse generator (IPG) configured to stimulate a vagus nerve, a hypoglossal nerve, a sacral nerve, one or more pelvic parasympathetic nerves, one or more lumbar sympathetic nerves, one or more pudendal nerves, or one or more peripheral nerves, of the subject; or
b) a pump configured to deliver insulin, baclofen, ziconotide, clonidine, bupivacaine, or an opioid to a bloodstream, organ, tissue, or any other body cavity or region of a body of the subject.

4. The stimulation system of claim 1, wherein the electronic device comprises a smart watch.

5. The stimulation system of claim 1, wherein the one or more implantable sensors comprise: an accelerometer, an inertial measurement unit (“IMU”), a magnetometer, a gyroscope, an electrocardiogram (“ECG”) sensor, a blood oxygen saturation (“SpO₂”) sensor, a blood pressure sensor, a glucometer, and/or a drug concentration sensor.

6. The stimulation system of claim 1, wherein the one or more external sensors comprise: an accelerometer, an IMU, a magnetometer, a gyroscope, an ECG sensor, an SpO₂ sensor, a blood pressure, sensor, a glucometer, or a drug concentration sensor.

7. The stimulation system of claim 6, wherein one or more of the one or more external sensors are integrated into, affixed to, or placed on, a watch, a ring, a bracelet, a band, a necklace, an earring, or an article of clothing.

8. The stimulation system of claim 1, wherein the electronic device is further configured to receive:
a) manual input from the subject, via a graphical user interface (“GUI”) provided by the electronic device, wherein the input comprises one or more parameters for the delivery of the electrical stimulation or active agent; and/or
b) input received from a remote server, comprising one or more parameters for the delivery of the electrical stimulation or active agent.

9. The stimulation system of claim 1, wherein the electronic device is configured to set or control one or more parameters of the delivery of the electrical stimulation to a nerve of a subject by setting or adjusting one or more parameters of the electrical stimulation, wherein the parameters comprise a pulse frequency, width, amplitude, and/or duty cycle, of the electrical stimulation.

10. The stimulation system of claim 1, wherein the electronic device is configured to set or control one or more parameters of the delivery of the active agent to one or more cells, tissues, or organs of the subject, by setting or adjusting one or more parameters, wherein the one or more parameters comprise activation of a pump, deactivation of the pump, a pumping duration, a pumping rate, a volume of active agent to deliver by the pump, and/or a timing schedule for pumping.

11. The stimulation system of claim 1, wherein the electronic device is further configured to:
determine one or more biometric parameters of the subject, based on the signal received from the at least one implantable sensor and/or the signal received from the at least one external sensor; and to
activate, terminate, titrate, or adjust the delivery of the electrical stimulation or active agent by the implantable stimulator, based on the determined one or more biometric parameters.

12. The stimulation system of claim 1, wherein the electronic device is further configured to:
determine one or more biometric parameters of the subject, based on the signal received from the at least one implantable sensor and/or the signal received from the at least one external sensor;
determine that the subject is experiencing, or is likely to experience, a medical condition or event, based on the determined one or more biometric parameters of the subject; and to
activate, terminate, titrate, or adjust, the delivery of the electrical stimulation or active agent by the implantable stimulator in response to determining that the subject is experiencing, or is likely to experience, a medical condition or event.

13. The stimulation system of claim 12, wherein the medical condition or event is tachycardia, an apnea or hypopnea event, a bradycardia event, a potential opioid overdose, or a hypoglycemia event.

14. The stimulation system of claim 1, wherein the implantable stimulator comprising a housing and wherein the one or more implantable sensors include one or more sensors integrated into, affixed to, or placed on the housing; and
wherein the implantable stimulator is configured to turn off one or more of the one or more sensors integrated into, affixed to, or placed on the housing in response to (a) a command transmitted by the electronic device, (b) determining that a wireless communication link has been established with the electronic device, or (c) determining that the electronic device is within a preset distance of or in proximity to the implantable stimulator.

15. The stimulation system of claim 1, wherein the system is configured to allow recalibration of the one or more implantable sensors using sensor data obtained from the one or more external sensors.

16. The stimulation system of claim 1, wherein the implantable controller is further configured to:
measure or receive a signal quality parameter for each of the one or more internal sensors and the one or more external sensors;
select a subset of sensors, comprising at least one of the one or more internal sensors and/or the one or more external sensors, based on the measured or received signal quality parameter(s);
deliver the electrical stimulation and/or active agent based on sensor data obtained from the subset of sensors.

17. A method of treating a subject, comprising:
providing a medical device comprising:
an implantable stimulator configured to deliver (a) electrical stimulation to a nerve of a subject; and/or (b) an active agent to one or more cells, tissues, or organs of the subject, and
an implantable controller, configured to control the implantable stimulator;
receiving, by the implantable controller, one or more stimulation parameters from an electronic device via a wireless communication link;
delivering the electrical stimulation or the active agent, by the implantable stimulator, based on the received one or more stimulation parameters, wherein the one or more stimulation parameters are based on:
(i) one or more biometric parameters of the subject determined using sensor data collected from one or more implantable sensors and one or more external sensors,
(ii) user input from the subject, entered via a graphical user interface (“GUI”) provided by the electronic device, wherein the input comprises one or more parameters for the delivery of the electrical stimulation or active agent; and/or
(iii) input received from a remote clinician, comprising one or more parameters for the delivery of the electrical stimulation or active agent.

18. The method of claim 17, wherein the electronic device comprises:
a) a wearable device, optionally a watch, a ring, a bracelet, a band, a necklace, or an earring;
b) a stationary device, optionally configured to be operated when the stationary device is placed on a surface; or
c) a portable device, optionally comprising an external housing configured to be held or carried by the subject.

19. The method of claim 17, wherein the implantable stimulator comprises:
a) an implantable pulse generator (“IPG”) configured to stimulate a vagus nerve, a hypoglossal nerve, a sacral nerve, one or more pelvic parasympathetic nerves, one or more lumbar sympathetic nerves, one or more pudendal nerves, or one or more peripheral nerves, of the subject; or
b) a pump configured to deliver insulin, baclofen, ziconotide, clonidine, bupivacaine, or an opioid to a bloodstream, organ, tissue, or any other body cavity or region of a body of the subject.

20. The method of any claim 17, wherein the one or more implantable sensors comprise an accelerometer, an inertial measurement unit (“IMU”), a magnetometer, a gyroscope, an electrocardiogram (“ECG”) sensor, a blood oxygen saturation (“SpO₂”) sensor, a blood pressure sensor, a glucometer, and/or a drug concentration sensor; and/or
the one or more external sensors comprise: an accelerometer, an IMU, a magnetometer, a gyroscope, an ECG sensor, an SpO₂ sensor, a blood pressure, sensor, a glucometer, or a drug concentration sensor.

21. The method of claim 17, wherein the one or more stimulation parameters received from the electronic device cause the implantable controller to:
a) begin, terminate, or titrate the delivery of the electrical stimulation or active agent;
b) set or adjust a pulse frequency, width, amplitude, and/or duty cycle, of the electrical stimulation; or
c) activate or deactivate the pump, to pump for a preset duration of time, to pump until a preset volume of active agent has been delivered, to pump according to a timing schedule.

22. The method of claim 17, wherein the electronic device comprises a smart watch.
